SEQUENCE LISTING (127 sequences)

SEQ ID NO: 1 - DNA, 20 bp, synthetic construct
  source 1..20: mol_type = other DNA
ttggagaactggatgtagat

SEQ ID NO: 2 - DNA, 20 bp, synthetic construct
  source 1..20: mol_type = other DNA
tggttggagaactggatgta

SEQ ID NO: 3 - DNA, 20 bp, synthetic construct
  source 1..20: mol_type = other DNA
ttgtggttggagaactggat

SEQ ID NO: 4 - DNA, 20 bp, synthetic construct
  source 1..20: mol_type = other DNA
tgtggttggagaactggatg

SEQ ID NO: 5 - DNA, 20 bp, synthetic construct
  source 1..20: mol_type = other DNA
gtggttggagaactggatgt

SEQ ID NO: 6 - DNA, 20 bp, synthetic construct
  source 1..20: mol_type = other DNA
aactggatgtagatgggctg

SEQ ID NO: 7 - DNA, 20 bp, synthetic construct
  source 1..20: mol_type = other DNA
ggttggagaactggatgtag

SEQ ID NO: 8 - DNA, 20 bp, synthetic construct
  source 1..20: mol_type = other DNA
aactggatgtagatgggctg

SEQ ID NO: 9 - DNA, 20 bp, synthetic construct
  source 1..20: mol_type = other DNA
ggttggagaactggatgtag

SEQ ID NO: 10 - DNA, 19 bp, synthetic construct
  source 1..19: mol_type = other DNA
gtggttggagaactggatg

SEQ ID NO: 11 - DNA, 20 bp, synthetic construct
  source 1..20: mol_type = other DNA
gaactggttgttcttggtga

SEQ ID NO: 12 - DNA, 20 bp, synthetic construct
  source 1..20: mol_type = other DNA
ctggaactggttgttcttgg

SEQ ID NO: 13 - DNA, 20 bp, synthetic construct
  source 1..20: mol_type = other DNA
ggaactggttgttcttggtg

SEQ ID NO: 14 - DNA, 20 bp, synthetic construct
  source 1..20: mol_type = other DNA
aactggttgttcttggtgaa

SEQ ID NO: 15 - DNA, 20 bp, synthetic construct
  source 1..20: mol_type = other DNA
gcctggaactggttgttctt

SEQ ID NO: 16 - DNA, 20 bp, synthetic construct
  source 1..20: mol_type = other DNA
cagcaggcgttgtagatgtt

SEQ ID NO: 17 - DNA, 20 bp, synthetic construct
  source 1..20: mol_type = other DNA
cgatgcgcagcgtgcagcag

SEQ ID NO: 18 - DNA, 20 bp, synthetic construct
  source 1..20: mol_type = other DNA
agcgtgcagcaggcgttgta

SEQ ID NO: 19 - DNA, 20 bp, synthetic construct
  source 1..20: mol_type = other DNA
agcaggcgttgtagatgttc

SEQ ID NO: 20 - DNA, 20 bp, synthetic construct
  source 1..20: mol_type = other DNA
cgcagcgtgcagcaggcgtt

SEQ ID NO: 21 - DNA, 20 bp, synthetic construct
  source 1..20: mol_type = other DNA
gcagcaggcgttgtagatgt

SEQ ID NO: 22 - DNA, 20 bp, synthetic construct
  source 1..20: mol_type = other DNA
caggcgttgtagatgttctg

SEQ ID NO: 23 - DNA, 20 bp, synthetic construct
  source 1..20: mol_type = other DNA
gcgcagcgtgcagcaggcgt

SEQ ID NO: 24 - DNA, 20 bp, synthetic construct
  source 1..20: mol_type = other DNA
tcgatgcgcagcgtgcagca

SEQ ID NO: 25 - DNA, 20 bp, synthetic construct
  source 1..20: mol_type = other DNA
agtcgatgcgcagcgtgcag

SEQ ID NO: 26 - DNA, 20 bp, synthetic construct
  source 1..20: mol_type = other DNA
gcgtgcagcaggcgttgtag

SEQ ID NO: 27 - DNA, 20 bp, synthetic construct
  source 1..20: mol_type = other DNA
cagcgtgcagcaggcgttgt

SEQ ID NO: 28 - DNA, 20 bp, synthetic construct
  source 1..20: mol_type = other DNA
gcaggcgttgtagatgttct

SEQ ID NO: 29 - DNA, 20 bp, synthetic construct
  source 1..20: mol_type = other DNA
tgcagcaggcgttgtagatg

SEQ ID NO: 30 - DNA, 20 bp, synthetic construct
  source 1..20: mol_type = other DNA
gcaggcgttgtagatgttct

SEQ ID NO: 31 - DNA, 20 bp, synthetic construct
  source 1..20: mol_type = other DNA
gtcgatgcgcagcgtgcagc

SEQ ID NO: 32 - DNA, 19 bp, synthetic construct
  source 1..19: mol_type = other DNA
cagcaggcgttgtagatgt

SEQ ID NO: 33 - DNA, 20 bp, synthetic construct
  source 1..20: mol_type = other DNA
cctgcatacggagaggctga

SEQ ID NO: 34 - DNA, 20 bp, synthetic construct
  source 1..20: mol_type = other DNA
tcctgcatacggagaggctg

SEQ ID NO: 35 - DNA, 20 bp, synthetic construct
  source 1..20: mol_type = other DNA
gctcctgcatacggagaggc

SEQ ID NO: 36 - DNA, 18 bp, synthetic construct
  source 1..18: mol_type = other DNA
ctttggggtgtgactctc

SEQ ID NO: 37 - DNA, 19 bp, synthetic construct
  source 1..19: mol_type = other DNA
gctttggggtgtgactctc

SEQ ID NO: 38 - DNA, 20 bp, synthetic construct
  source 1..20: mol_type = other DNA
ggctttggggtgtgactctc

SEQ ID NO: 39 - DNA, 20 bp, synthetic construct
  source 1..20: mol_type = other DNA
gctttggggtgtgactctct

SEQ ID NO: 40 - DNA, 26 bp, synthetic construct
  source 1..26: mol_type = other DNA
gctttggggtgtgtttttttctctct

SEQ ID NO: 41 - DNA, 27 bp, synthetic construct
  source 1..27: mol_type = other DNA
gctttggggtgtggggttcccctctct

SEQ ID NO: 42 - DNA, 28 bp, synthetic construct
  source 1..28: mol_type = other DNA
gctttggggtgtggcctttggcctctct

SEQ ID NO: 43 - DNA, 28 bp, synthetic construct
  source 1..28: mol_type = other DNA
gctttggggtgtcgcctttggcgtctct

SEQ ID NO: 44 - DNA, 29 bp, synthetic construct
  source 1..29: mol_type = other DNA
gctttggggtgtgcgcctttggcgtctct

SEQ ID NO: 45 - DNA, 29 bp, synthetic construct
  source 1..29: mol_type = other DNA
gctttggggtgtgttttttttttttctct

SEQ ID NO: 46 - DNA, 34 bp, synthetic construct
  source 1..34: mol_type = other DNA
gctttggggtgtggggtgggttgggtgggtctct

SEQ ID NO: 47 - DNA, 35 bp, synthetic construct
  source 1..35: mol_type = other DNA
gctttggggtgtggggtgggttgggtgggctctct

SEQ ID NO: 48 - DNA, 40 bp, synthetic construct
  source 1..40: mol_type = other DNA
gctttggggtgtggggttgggtttgggttgggttctctct

SEQ ID NO: 49 - DNA, 42 bp, synthetic construct
  source 1..42: mol_type = other DNA
gctttggggtgtgttgggttgggtttgggttgggttctctct

SEQ ID NO: 50 - DNA, 40 bp, synthetic construct
  source 1..40: mol_type = other DNA
gctttggggtgtgtgcgtttcgctcgctttgcgtctctct

SEQ ID NO: 51 - DNA, 20 bp, synthetic construct
  source 1..20: mol_type = other DNA
aataaagaggctttggggtg

SEQ ID NO: 52 - DNA, 20 bp, synthetic construct
  source 1..20: mol_type = other DNA
ataaagaggctttggggtgt

SEQ ID NO: 53 - DNA, 20 bp, synthetic construct
  source 1..20: mol_type = other DNA
gaataaagaggctttggggt

SEQ ID NO: 54 - DNA, 20 bp, synthetic construct
  source 1..20: mol_type = other DNA
gaggctttggggtgtgactc

SEQ ID NO: 55 - DNA, 20 bp, synthetic construct
  source 1..20: mol_type = other DNA
aggctttggggtgtgactct

SEQ ID NO: 56 - DNA, 20 bp, synthetic construct
  source 1..20: mol_type = other DNA
agaggctttggggtgtgact

SEQ ID NO: 57 - DNA, 20 bp, synthetic construct
  source 1..20: mol_type = other DNA
aagaggctttggggtgtgac

SEQ ID NO: 58 - DNA, 20 bp, synthetic construct
  source 1..20: mol_type = other DNA
agaataaagaggctttgggg

SEQ ID NO: 59 - DNA, 16 bp, synthetic construct
  source 1..16: mol_type = other DNA
ggctttggggtgtgac

SEQ ID NO: 60 - DNA, 19 bp, synthetic construct
  source 1..19: mol_type = other DNA
gaggctttggggtgtgact

SEQ ID NO: 61 - DNA, 19 bp, synthetic construct
  source 1..19: mol_type = other DNA
aataaagaggctttggggt

SEQ ID NO: 62 - DNA, 20 bp, synthetic construct
  source 1..20: mol_type = other DNA
ttattgaacaggatcttcac

SEQ ID NO: 63 - DNA, 20 bp, synthetic construct
  source 1..20: mol_type = other DNA
cgttctccttcttattgaac

SEQ ID NO: 64 - DNA, 20 bp, synthetic construct
  source 1..20: mol_type = other DNA
gcgttctccttcttattgaa

SEQ ID NO: 65 - DNA, 20 bp, synthetic construct
  source 1..20: mol_type = other DNA
cttattgaacaggatcttca

SEQ ID NO: 66 - DNA, 20 bp, synthetic construct
  source 1..20: mol_type = other DNA
ctccttcttattgaacagga

SEQ ID NO: 67 - DNA, 20 bp, synthetic construct
  source 1..20: mol_type = other DNA
ccttcttattgaacaggatc

SEQ ID NO: 68 - DNA, 20 bp, synthetic construct
  source 1..20: mol_type = other DNA
gttctccttcttattgaaca

SEQ ID NO: 69 - DNA, 20 bp, synthetic construct
  source 1..20: mol_type = other DNA
tctccttcttattgaacagg

SEQ ID NO: 70 - DNA, 16 bp, synthetic construct
  source 1..16: mol_type = other DNA
gttctccttcttattg

SEQ ID NO: 71 - DNA, 20 bp, synthetic construct
  source 1..20: mol_type = other DNA
ccgttcaggtggctcatggc

SEQ ID NO: 72 - DNA, 20 bp, synthetic construct
  source 1..20: mol_type = other DNA
cgttcaggtggctcatggcc

SEQ ID NO: 73 - DNA, 26 bp, synthetic construct
  source 1..26: mol_type = other DNA
gcgttttttttttccttcttattgaa

SEQ ID NO: 74 - DNA, 30 bp, synthetic construct
  source 1..30: mol_type = other DNA
gcgttcgcctttggcgtccttcttattgaa

SEQ ID NO: 75 - DNA, 26 bp, synthetic construct
  source 1..26: mol_type = other DNA
gcgttctccttctttttttttttgaa

SEQ ID NO: 76 - DNA, 30 bp, synthetic construct
  source 1..30: mol_type = other DNA
gcgttctccttcttcgcctttggcgttgaa

SEQ ID NO: 77 - DNA, 29 bp, synthetic construct
  source 1..29: mol_type = other DNA
gcgttctccttctcgcctttggcgttgaa

SEQ ID NO: 78 - DNA, 29 bp, synthetic construct
  source 1..29: mol_type = other DNA
gcgttctcccgcctttggcgcttattgaa

SEQ ID NO: 79 - DNA, 29 bp, synthetic construct
  source 1..29: mol_type = other DNA
gcgttctccggcctttggcccttattgaa

SEQ ID NO: 80 - DNA, 25 bp, synthetic construct
  source 1..25: mol_type = other DNA
gcgttctcctttttttcttattgaa

SEQ ID NO: 81 - DNA, 20 bp, synthetic construct
  source 1..20: mol_type = other DNA
cgtgcagcttgtgcccgttc

SEQ ID NO: 82 - DNA, 20 bp, synthetic construct
  source 1..20: mol_type = other DNA
gtgcagcttgtgcccgttca

SEQ ID NO: 83 - DNA, 20 bp, synthetic construct
  source 1..20: mol_type = other DNA
cccgtgcagcttgtgcccgt

SEQ ID NO: 84 - DNA, 20 bp, synthetic construct
  source 1..20: mol_type = other DNA
ccgtgcagcttgtgcccgtt

SEQ ID NO: 85 - DNA, 20 bp, synthetic construct
  source 1..20: mol_type = other DNA
tcccgtgcagcttgtgcccg

SEQ ID NO: 86 - DNA, 20 bp, synthetic construct
  source 1..20: mol_type = other DNA
gtgcccgttcaggtggctca

SEQ ID NO: 87 - DNA, 20 bp, synthetic construct
  source 1..20: mol_type = other DNA
tgcagcttgtgcccgttcag

SEQ ID NO: 88 - DNA, 20 bp, synthetic construct
  source 1..20: mol_type = other DNA
ttgtgcccgttcaggtggct

SEQ ID NO: 89 - DNA, 20 bp, synthetic construct
  source 1..20: mol_type = other DNA
cttgtgcccgttcaggtggc

SEQ ID NO: 90 - DNA, 20 bp, synthetic construct
  source 1..20: mol_type = other DNA
tgcccgttcaggtggctcat

SEQ ID NO: 91 - DNA, 20 bp, synthetic construct
  source 1..20: mol_type = other DNA
ttcccgtgcagcttgtgccc

SEQ ID NO: 92 - DNA, 20 bp, synthetic construct
  source 1..20: mol_type = other DNA
cccgttcaggtggctcatgg

SEQ ID NO: 93 - DNA, 20 bp, synthetic construct
  source 1..20: mol_type = other DNA
cttcccgtgcagcttgtgcc

SEQ ID NO: 94 - DNA, 19 bp, synthetic construct
  source 1..19: mol_type = other DNA
ttcccgtgcagcttgtgcc

SEQ ID NO: 95 - DNA, 20 bp, synthetic construct
  source 1..20: mol_type = other DNA
gagggcgggatgttggagag

SEQ ID NO: 96 - DNA, 20 bp, synthetic construct
  source 1..20: mol_type = other DNA
agggcgggatgttggagagg

SEQ ID NO: 97 - DNA, 20 bp, synthetic construct
  source 1..20: mol_type = other DNA
gggcgggatgttggagaggt

SEQ ID NO: 98 - DNA, 20 bp, synthetic construct
  source 1..20: mol_type = other DNA
ggcgggatgttggagaggtg

SEQ ID NO: 99 - DNA, 20 bp, synthetic construct
  source 1..20: mol_type = other DNA
gcgggatgttggagaggtgc

SEQ ID NO: 100 - DNA, 20 bp, synthetic construct
  source 1..20: mol_type = other DNA
cgggatgttggagaggtgca

SEQ ID NO: 101 - DNA, 20 bp, synthetic construct
  source 1..20: mol_type = other DNA
tgccatcttgcggtccttct

SEQ ID NO: 102 - DNA, 20 bp, synthetic construct
  source 1..20: mol_type = other DNA
gcggtccttctggaagaact

SEQ ID NO: 103 - DNA, 19 bp, synthetic construct
  source 1..19: mol_type = other DNA
cggtccttctggaagaact

SEQ ID NO: 104 - DNA, 20 bp, synthetic construct
  source 1..20: mol_type = other DNA
cggtccttctggaagaactt

SEQ ID NO: 105 - DNA, 20 bp, synthetic construct
  source 1..20: mol_type = other DNA
ccatcttgcggtccttctgg

SEQ ID NO: 106 - DNA, 20 bp, synthetic construct
  source 1..20: mol_type = other DNA
catcttgcggtccttctgga

SEQ ID NO: 107 - DNA, 20 bp, synthetic construct
  source 1..20: mol_type = other DNA
atcttgcggtccttctggaa

SEQ ID NO: 108 - DNA, 20 bp, synthetic construct
  source 1..20: mol_type = other DNA
cttgcggtccttctggaaga

SEQ ID NO: 109 - DNA, 20 bp, synthetic construct
  source 1..20: mol_type = other DNA
ttgcggtccttctggaagaa

SEQ ID NO: 110 - DNA, 20 bp, synthetic construct
  source 1..20: mol_type = other DNA
tgcggtccttctggaagaac

SEQ ID NO: 111 - DNA, 20 bp, synthetic construct
  source 1..20: mol_type = other DNA
cagtgccatcttgcggtcct

SEQ ID NO: 112 - DNA, 20 bp, synthetic construct
  source 1..20: mol_type = other DNA
gtgccatcttgcggtccttc

SEQ ID NO: 113 - DNA, 20 bp, synthetic construct
  source 1..20: mol_type = other DNA
gatcagtgccatcttgcggt

SEQ ID NO: 114 - DNA, 20 bp, synthetic construct
  source 1..20: mol_type = other DNA
agtgccatcttgcggtcctt

SEQ ID NO: 115 - DNA, 20 bp, synthetic construct
  source 1..20: mol_type = other DNA
atcagtgccatcttgcggtc

SEQ ID NO: 116 - DNA, 20 bp, synthetic construct
  source 1..20: mol_type = other DNA
tggatcagtgccatcttgcg

SEQ ID NO: 117 - DNA, 20 bp, synthetic construct
  source 1..20: mol_type = other DNA
tcagtgccatcttgcggtcc

SEQ ID NO: 118 - DNA, 20 bp, synthetic construct
  source 1..20: mol_type = other DNA
ggatcagtgccatcttgcgg

SEQ ID NO: 119 - DNA, 20 bp, synthetic construct
  source 1..20: mol_type = other DNA
agcccatctggatcagtgcc

SEQ ID NO: 120 - DNA, 20 bp, synthetic construct
  source 1..20: mol_type = other DNA
gagcccatctggatcagtgc

SEQ ID NO: 121 - DNA, 20 bp, synthetic construct
  source 1..20: mol_type = other DNA
gcccatctggatcagtgcca

SEQ ID NO: 122 - DNA, 20 bp, synthetic construct
  source 1..20: mol_type = other DNA
agcccatctggatcagtgcc

SEQ ID NO: 123 - DNA, 20 bp, synthetic construct
  source 1..20: mol_type = other DNA
cccatctggatcagtgccat

SEQ ID NO: 124 - DNA, 20 bp, synthetic construct
  source 1..20: mol_type = other DNA
gtggaaatattgctaggcac

SEQ ID NO: 125 - DNA, 24 bp, synthetic construct
  source 1..24: mol_type = other DNA
gtggaaaaaaatattgctaggcac

SEQ ID NO: 126 - DNA, 20 bp, synthetic construct
  source 1..20: mol_type = other DNA
cctataggactatccaggaa

SEQ ID NO: 127 - DNA, 3206 bp, Homo sapiens
  source 1..3206: mol_type = genomic DNA
attccggcgcctccactccgtcccccgcgggtctgctctgtgtgccatggacggcattgtcccagatatagccgttggtacaaagcggggatctgacgagcttttctctacttgtgtcactaacggaccgtttatcatgagcagcaactcggcttctgcagcaaacggaaatgacagcaagaagttcaaaggtgacagccgaagtgcaggcgtcccctctagagtgatccacatccggaagctccccatcgacgtcacggagggggaagtcatctccctggggctgccctttgggaaggtcaccaacctcctgatgctgaaggggaaaaaccaggccttcatcgagatgaacacggaggaggctgccaacaccatggtgaactactacacctcggtgacccctgtgctgcgcggccagcccatctacatccagttctccaaccacaaggagctgaagaccgacagctctcccaaccaggcgcgggcccaggcggccctgcaggcggtgaactcggtccagtcggggaacctggccttggctgcctcggcggcggccgtggacgcagggatggcgatggccgggcagagccccgtgctcaggatcatcgtggagaacctcttctaccctgtgaccctggatgtgctgcaccagattttctccaagttcggcacagtgttgaagatcatcaccttcaccaagaacaaccagttccaggccctgctgcagtatgcggaccccgtgagcgcccagcacgccaagctgtcgctggacgggcagaacatctacaacgcctgctgcacgctgcgcatcgacttttccaagctcaccagcctcaacgtcaagtacaacaatgacaagagccgtgactacacacgcccagacctgccttccggggacagccagccctcgctggaccagaccatggccgcggccttcggtgcacctggtataatctcagcctctccgtatgcaggagctggtttccctcccacctttgccattcctcaagctgcaggcctttccgttccgaacgtccacggcgccctggcccccctggccatcccctcggcggcggcggcagctgcggcggcaggtcggatcgccatcccgggcctggcgggggcaggaaattctgtattgctggtcagcaacctcaacccagagagagtcacaccccaaagcctctttattcttttcggcgtctacggtgacgtgcagcgcgtgaagatcctgttcaataagaaggagaacgccctagtgcagatggcggacggcaaccaggcccagctggccatgagccacctgaacgggcacaagctgcacgggaagcccatccgcatcacgctctcgaagcaccagaacgtgcagctgccccgcgagggccaggaggaccagggcctgaccaaggactacggcaactcacccctgcaccgcttcaagaagccgggctccaagaacttccagaacatattcccgccctcggccacgctgcacctctccaacatcccgccctcagtctccgaggaggatctcaaggtcctgttttccagcaatgggggcgtcgtcaaaggattcaagttcttccagaaggaccgcaagatggcactgatccagatgggctccgtggaggaggcggtccaggccctcattgacctgcacaaccacgacctcggggagaaccaccacctgcgggtctccttctccaagtccaccatctaggggcacaggcccccacggccgggccccctggcgacaacttccatcattccagagaaaagccactttaaaaacagctgaagtgaccttagcagaccagagattttatttttttaaagagaaatcagtttacctgtttttaaaaaaattaaatctagttcaccttgctcaccctgcggtgacagggacagctcaggctcttggtgactgtggcagcgggagttcccggccctccacacccggggccagaccctcggggccatgccttggtggggcctgtgtcgggcgtggggcctgcaggtgggcgccccgaccacgacttggcttccttgtgccttaaaaaacctgccttcctgcagccacacacccacccggggtgtcctggggacccaaggggtgggggggtcacaccagagagaggcagggggcctggccggctcctgcaggatcatgcagctggggcgcggcggccgcggctgcgacaccccaaccccagccctctaatcaagtcacgtgattctcccttcaccccgcccccagggccttcccttctgcccccaggcgggctccccgctgctccagctgcggagctggtcgacataatctctgtattatatactttgcagttgcagacgtctgtgcctagcaatatttccagttgaccaaatattctaatcttttttcatttatatgcaaaagaaatagttttaagtaactttttatagcaagatgatacaatggtatgagtgtaatctaaacttccttgtggtattaccttgtatgctgttacttttattttattccttgtaattaagtcacaggcaggacccagtttccagagagcaggcggggccgcccagtgggtcaggcacagggagccccggtcctatcttagagcccctgagcttcagggaaggggcgggcgtgtcgccgcctctggcatcgcctccggttgccttacaccacgccttcacctgcagtcgcctagaaaacttgctctcaaacttcagggttttttcttccttcaaattttggaccaaagtctcatttctgtgttttgcctgcctctgatgctgggacccggaaggcgggcgctcctcctgtcttctctgtgctctttctaccgcccccgcgtcctgtcccgggggctctcctaggatcccctttccgtaaaagcgtgtaacaagggtgtaaatatttataattttttatacctgttgtgagacccgaggggcggcggcgcggttttttatggtgacacaaatgtatattttgctaacagcaattccaggctcagtattgtgaccgcggagccacaggggaccccacgcacattccgttgccttacccgatggcttgtgacgcggagagaaccgattaaaaccgtttgagaaactcctcccttgtctagccctgtgttcgctgtggacgctgtagaggcaggttggccagtctgtacctggacttcgaataaatcttctgtatcctc

(19) United States
(12) Patent Application Publication
(10) Pub. No.: US 2026/0218185 A1
(43) Pub. Date: Jul. 30, 2026

(54) ANTISENSE OLIGONUCLEOTIDE TARGETING POLYPYRIMIDINE TRACT BINDING PROTEIN AND USE THEREOF

(71) Applicant: LNCTAC CO., LTD, Chengdu, Shuangliu District (CN)

(72) Inventors: Haodong CHEN, Chengdu (CN); Wanwan Li, Chengdu (CN); Grimm MAXIMILIAN, Chengdu (CN); Lina WU, Chengdu (CN); Ting KANG, Chengdu (CN); Lei ZHANG, Chengdu (CN); Chong YUAN, Chengdu (CN)

(21) Appl. No.: 19/147,189

(22) PCT Filed: Jan. 10, 2024
(86) PCT No.: PCT/CN2024/071522

§ 371 Date: Nov. 13, 2025

(30) Foreign Application Priority Data
Jan. 10, 2023 (CN) 202310037334.5

(51) Int. Cl. C12N 15/113

(52) U.S. Cl. CPC C12N 15/113; C12N 2310/11; C12N 2310/321

(57) ABSTRACT

The present disclosure related to antisense oligonucleotide capable of specifically targeting the mRNA sequence of a polypyrimidine tract binding protein 1 (PTBP1) and the use thereof. The antisense oligonucleotide can reduce the expression of PTBP1, thereby treating diseases caused by abnormal expression of the PTBP1 gene.

### TECHNICAL FIELD

The disclosure relates to biomedicine, specifically to antisense oligonucleotides targeting polypyrimidine region-binding proteins and use thereof.

### BACKGROUND

Malignant tumors have become one of the most common serious threats to human life and quality of life. In recent years, targeted therapy, which involves blocking various tumor-promoting factors in the human body, has emerged as a mainstream direction in cancer drug research. It has become another highly effective method of treating cancer, alongside surgery, radiation therapy, and chemotherapy. Targeted therapy has achieved significant progress in clinical applications and demonstrated important clinical value.

Gene-targeted therapy at the mRNA level is a method of targeted therapy that is currently a popular area of development. This therapy functions by inducing endogenous splicing or inhibiting the expression of disease-causing genes in patients. It has already achieved notable progress. Currently, available gene therapy products primarily target rare diseases, gradually expanding into common conditions. This suggests that gene therapy could be a potential treatment for cancer. Antisense oligonucleotides, which specifically inhibit mRNA expression and reduce regulated protein expression, are one of the most competitive technologies in targeted gene therapy.

Regulation of epigenetic effects some endogenous genes or proteins act as regulators of post-transcriptional gene expression. They participate in multiple functions, such as mRNA splicing, translation, stability, and colocalization, and play a crucial role in the cell cycle. However, overexpressing these genes or proteins is also considered an important factor in promoting tumorigenesis. The polypyrimidine tract binding protein 1 (PTBP1) gene belongs to the RNA-binding protein (RBP) nuclear heterogeneous ribonucleoprotein (hnRNP) family. PTBP1 participates in regulating various post-transcriptional modification processes, including alternative splicing. PTBP1 participates in various cellular activities, such as neuronal growth and differentiation, T-cell activation, spermatogenesis, embryonic development, and erythrocyte development. PTBP1 plays a crucial role throughout the developmental process of neuronal cells, including transcription, neurogenesis, synapse maturation, and differentiation. PTBP1 is highly expressed in various tumor cells and can influence tumor progression by affecting tumor tissue energy metabolism, promoting tumor development, and regulating tumor autophagy, apoptosis, epithelial-mesenchymal transition, and tumor immunity. Reducing PTBP1 expression at the mRNA level and inhibiting its gene regulatory protein expression may be a way to treat various malignant tumors. Literature reports indicate that PTBP1 promotes tumor cell proliferation and migration by activating pathways such as HIF1α. PTBP1 inhibitors can overcome tumor resistance and enhance tumor drug sensitivity. Furthermore, PTBP1 inhibition has been reported to treat Parkinson's disease, Alzheimer's disease, Huntington's disease, and other central nervous system (CNS) disorders.

Currently, there are no studies on antisense oligonucleotides targeting the inhibition of PTBP1 mRNA in human cells.

### SUMMARY

Firstly, the present disclosure provides a group of antisense oligonucleotide (ASO) molecules that specifically target the following regions of the PTBP1 mRNA sequence:
EXON5 positions 416-446;
EXON7 positions 692-718;
EXON8 positions 776-815;
EXON9 positions 956-979;
EXON10/EXON11 positions 1160-1194;
EXON12 positions 1223-1255;
EXON12/EXON13 positions 1291-1324;
EXON13 positions 1305-1330;
EXON13/EXON14 positions 1491-1516;
EXON14/EXON15 positions 1577-1605;
EXON15 positions 1587-1624;
EXON15 3′UTR positions 2356-2376.

Preferably, the PTBP1 mRNA sequence is as shown in SEQ ID NO: 127.

More preferably, the ASO molecules specifically target the following regions of the PTBP1 mRNA sequence:
EXON10/EXON11 positions 1164-1188;
EXON5 positions 416-436;
EXON8 positions 788-815;
EXON12/EXON13 positions 1299-1324;
EXON15 positions 1587-1624.

Most preferably, the ASO molecules target EXON10/EXON11 positions 1164-1185.

Furthermore, the molecular lengths of the ASO molecules are 15 to 20 nucleotides;

Preferably, the ASO molecules are selected from at least one of the nucleotide sequences shown in SEQ ID NO: 1-123.

More preferably, the ASO molecules are selected from SEQ ID NO: 6, 20, 31, 36-41, 55-56, 60, 87, 89, 100-101, 116-119, or 123.

Most preferably, the ASO molecules are selected from SEQ ID NO: 38 or 39.

Further, the ASO molecules include at least one modified nucleotide.

Preferably, the modified nucleotide is selected from one or more of the following: 5′-thio-phosphate-modified nucleotides, 5-methylated cytosine nucleotides, 2′-O-methyl-modified nucleotides, 2′-O-2-methoxyethyl-modified nucleotides, 2′-fluorinated nucleotides, 3′-nitrogen-substituted nucleotides, 2′-deoxy-2′-fluorinated nucleotides, 2′-deoxy-modified nucleotides, locked nucleotides, dealkylated nucleotides, 2′-amino-modified nucleotides, morpholine-substituted nucleotides, peptide nucleotides, and amino phosphates;

Most preferably, the modification refers to all nucleotides in the ASO sequence being 5′-thio-phosphate-substituted nucleotides, with the first 5 nucleotides at both the 5′ and 3′ ends modified with 2′-O-methoxyethyl (MOE).

The present disclosure also relates to drugs or pharmaceutical compositions containing the ASO molecule, wherein the drugs or drug compositions contain a therapeutically effective amount of the ASO molecule and necessary pharmaceutical excipients.

Preferably, the drug or pharmaceutical compositions can be prepared into different formulations using conventional methods. For example, physiological saline or water-soluble solvents containing other excipients can be used to prepare injections using conventional methods, and the drug or pharmaceutical compositions can be administered via various routes, including local, intravenous, intramuscular, subcutaneous, or intradermal.

The present disclosure further relates to the use of the ASO molecules, drugs, or pharmaceutical compositions in inhibiting PTBP1 gene expression.

The present disclosure also relates to the use of the ASO molecules in the preparation of drugs, wherein the drugs are used to treat diseases caused by abnormal expression of the PTBP1 gene; the diseases include, but are not limited to, tumors, Parkinson's disease, Alzheimer's disease, and Huntington's disease; preferably, the disease is a tumor.

### Terms and Definition

Unless otherwise defined, all scientific and technical terms mentioned in this specification have the same meanings as those commonly understood by those skilled in the art. To facilitate a better understanding of the invention, the following terms are explained below.

The terms used in this invention are intended solely to describe specific embodiments and do not intend to limit the scope of the invention. Unless otherwise explicitly indicated by the context, the singular forms “one,” “a,” and “the” used in this invention also intend to include plural forms. Furthermore, the open-ended expressions “including” and “comprising” are interpreted as also including structural components or method steps not explicitly mentioned, but it should be noted that these open-ended expressions also cover situations where the invention consists solely of the components and method steps explicitly mentioned (i.e., they cover situations described by the closed-ended expression ‘comprising’). As used throughout the invention, the term “range” is used as a shorthand form to describe each value and all values within that range. Any value within the range, such as an integer value, may be selected as the endpoint of the range. For example, the integer representation of the range 2-13 includes the integers 2, 3, 4, 5, 6, 7, 8, 9, 10, 11, 12, and 13, as well as any subranges formed by them, such as 2-10, 3-12, 4-9, etc.

In the present invention, the terms “nucleic acid,” “nucleic acid molecule,” “oligonucleotide drug,” and “nucleic acid drug” are interchangeable and refer to any DNA, RNA, or DNA/RNA chimera, and may be oligonucleotides or polynucleotides, and may be unmodified RNA or DNA or modified RNA or DNA. These terms include, but are not limited to, single-stranded and double-stranded DNA, DNA comprising a mixture of single-stranded and double-stranded regions, single-stranded and double-stranded RNA, RNA comprising a mixture of single-stranded and double-stranded regions, and hybrid molecules comprising DNA and RNA that may be single-stranded or double-stranded or a mixture of single-stranded and double-stranded regions.

In some embodiments, the nucleic acid may contain one or more modified nucleotides, modified bonds, etc.

Examples of modified bonds or inter-nucleotide bonds suitable for the present invention include thiophosphate esters, dithiophosphate esters, etc. Other nucleic acids containing phosphorus atoms that are suitable for modification include, for example, thiophosphate esters, chiral thiophosphate esters, dithiophosphate esters, phosphotriesters, aminoalkyl triphosphate esters, methyl and other alkyl phosphate esters including 3′-alkyl phosphate esters, 5′-alkyl phosphate esters, and chiral phosphate esters, phosphonates, amino phosphates including 3′-aminoamino phosphates and aminoalkylamino phosphates, diamino phosphates, thiocarbonylamino phosphates, thiocarbonylalkyl phosphates, thioalkyl phosphate triesters, selenium-substituted phosphates and boron-substituted phosphates with normal 3′-5′ bonds; wherein one or more nucleotide inter-bonds are 3′ to 3′, 5′ to 5′, or 2′ to 2′ bonds.

Nucleotide modifications suitable for the present invention also include sugar substituents selected from the following: OH; F; O—, S—, or N-alkyl; O—, S—, or N-alkene; O—, S—, or N-alkyne; or O-alkyl-O-alkyl, wherein the alkyl, alkene, and alkyne groups may be substituted or unsubstituted C1 to C10 alkyl or C2 to C10 alkene and alkyne groups. Particularly suitable are O((CH₂)_nO)_mCH₃, O(CH₂)_nOCH₃, O(CH₂)_nNH₂, O(CH₂)_nCH₃, O(CH₂)_nONH₂, and O(CH₂)_nON ((CH₂)_nCH₃)₂, where n and m are 1 to about 10. Other suitable sugar substituents include methoxy (—O—CH₃), amino propoxy (—OCH₂CH₂CH₂NH₂), allyl (—CH₂—CH═CH₂), —O-allyl (—O—CH₂—CH═CH₂), and fluorine (F). The 2′-sugar substituent may be located at the arabinose (upper) position or the ribose (lower) position. The technique for modifying nucleotides is well known in the art and need not be described herein.

The “unmodified” or “natural” nucleotides used in the present invention include adenine (A), guanine (G), thymine (T), cytosine (C), and uracil (U).

The nucleic acid molecules of the present invention and the target nucleic acids protected by the nucleic acid molecules of the present invention, such as nucleic acid drugs, can be used for various applications, such as gene knockdown, gene knockout, gene activation, gene modification, gene editing, gene regulation, protein expression, protein regulation, or bioassays, or as nucleic acid drugs. Therefore, the present invention anticipates the use of the nucleic acid molecules of the present invention and the target nucleic acids protected by the nucleic acid molecules of the present invention in this regard, such as for the preparation of drugs for the aforementioned applications.

In the present invention, the terms “conjugated” and “linked” are interchangeable and refer to connection via chemical bonds.

In the present invention, the stability of the nucleic acids described herein in serum refers to the stability of the nucleic acids in various concentrations of serum, such as 50% serum. In the present invention, the improvement in stability refers to an increase in the stability of the target nucleic acid, such as its stability in serum, compared to the target nucleic acid that has not been protected by the nucleic acid molecules of the present invention or by other methods. In the present invention, the improvement in the activity of the target nucleic acid refers to an increase in the biological activity of the target nucleic acid after being protected by the nucleic acid molecules of the present invention, compared to the target nucleic acid that has not been protected by the nucleic acid molecules of the present invention or by other methods. In some embodiments, the biological activity is selected from gene knockdown activity, gene knockout activity, gene activation activity, gene modification activity, gene editing activity, gene regulation activity, protein expression activity, protein regulation activity, or bioassay activity, or activity for use as a nucleic acid drug, etc. In some embodiments, the activity of the target nucleic acid is equivalent to or similar to the activity of the target nucleic acid that has not been protected by the nucleic acid molecules of the present invention or by other methods, but its stability is improved.

The term “pharmaceutically acceptable carrier” as used herein is intended to include any and all solvents, dispersing media, coating agents, antibacterial and antifungal agents, isotonic agents, and absorption delay agents compatible with drug delivery. Suitable carriers are described in the latest edition of Remington's Pharmaceutical Sciences, which is incorporated herein by reference. Preferred examples of such carriers or diluents include, but are not limited to, water, saline, Ringer's solution, dextrose solution, and 5% human serum albumin. The use of such media and reagents is well known in the art. Unless any conventional medium or reagent is incompatible with the substances provided by the present invention, their use in the composition is considered.

The following specific examples are provided to illustrate the scheme of the present invention, but they are not intended to limit the technical scheme of the present invention. Those skilled in the art should understand that modifications or equivalent replacements may still be made to the invention without departing from the spirit and scope of the present invention, and any such modifications or partial replacements should be included within the scope of protection of the present invention.

### DESCRIPTION OF THE FIGURES

FIG. 1 Inhibition of PTBP1 protein expression in Hela cells after treatment with antisense oligonucleotides or their modified forms shown in Table 1;

FIG. 2 Inhibition of cell proliferation in HCT116 cells after treatment with antisense oligonucleotides shown in Table 1;

### DETAILED DESCRIPTION

### Example 1, Design, Synthesis, and Modification of ASOs Targeting PTBP1

The PTBP1 mRNA sequence (ENST00000356948.11) from the Ensembl database was selected as the target, with the sequence shown in SEQ ID NO: 127.

SEQ ID NO. 127:
ATTCCGGCGCCTCCACTCCGTCCCCCGCGGGTCTGCTCTGTGTGCCATGGACGGC
ATTGTCCCAGATATAGCCGTTGGTACAAAGCGGGGATCTGACGAGCTTTTCTCTACTT
GTGTCACTAACGGACCGTTTATCATGAGCAGCAACTCGGCTTCTGCAGCAAACGGAA
ATGACAGCAAGAAGTTCAAAGGTGACAGCCGAAGTGCAGGCGTCCCCTCTAGAGTG
ATCCACATCCGGAAGCTCCCCATCGACGTCACGGAGGGGGAAGTCATCTCCCTGGGG
CTGCCCTTTGGGAAGGTCACCAACCTCCTGATGCTGAAGGGGAAAAACCAGGCCTTC
ATCGAGATGAACACGGAGGAGGCTGCCAACACCATGGTGAACTACTACACCTCGGTG
ACCCCTGTGCTGCGCGGCCAGCCCATCTACATCCAGTTCTCCAACCACAAGGAGCTG
AAGACCGACAGCTCTCCCAACCAGGCGCGGGCCCAGGCGGCCCTGCAGGCGGTGAA
CTCGGTCCAGTCGGGGAACCTGGCCTTGGCTGCCTCGGCGGCGGCCGTGGACGCAG
GGATGGCGATGGCCGGGCAGAGCCCCGTGCTCAGGATCATCGTGGAGAACCTCTTCT
ACCCTGTGACCCTGGATGTGCTGCACCAGATTTTCTCCAAGTTCGGCACAGTGTTGA
AGATCATCACCTTCACCAAGAACAACCAGTTCCAGGCCCTGCTGCAGTATGCGGACC
CCGTGAGCGCCCAGCACGCCAAGCTGTCGCTGGACGGGCAGAACATCTACAACGCC
TGCTGCACGCTGCGCATCGACTTTTCCAAGCTCACCAGCCTCAACGTCAAGTACAAC
AATGACAAGAGCCGTGACTACACACGCCCAGACCTGCCTTCCGGGGACAGCCAGCC
CTCGCTGGACCAGACCATGGCCGCGGCCTTCGGTGCACCTGGTATAATCTCAGCCTCT
CCGTATGCAGGAGCTGGTTTCCCTCCCACCTTTGCCATTCCTCAAGCTGCAGGCCTTT
CCGTTCCGAACGTCCACGGCGCCCTGGCCCCCCTGGCCATCCCCTCGGCGGCGGCGG
CAGCTGCGGCGGCAGGTCGGATCGCCATCCCGGGCCTGGCGGGGGCAGGAAATTCT
GTATTGCTGGTCAGCAACCTCAACCCAGAGAGAGTCACACCCCAAAGCCTCTTTATT
CTTTTCGGCGTCTACGGTGACGTGCAGCGCGTGAAGATCCTGTTCAATAAGAAGGAG
AACGCCCTAGTGCAGATGGCGGACGGCAACCAGGCCCAGCTGGCCATGAGCCACCT
GAACGGGCACAAGCTGCACGGGAAGCCCATCCGCATCACGCTCTCGAAGCACCAGA
ACGTGCAGCTGCCCCGCGAGGGCCAGGAGGACCAGGGCCTGACCAAGGACTACGGC
AACTCACCCCTGCACCGCTTCAAGAAGCCGGGCTCCAAGAACTTCCAGAACATATTC
CCGCCCTCGGCCACGCTGCACCTCTCCAACATCCCGCCCTCAGTCTCCGAGGAGGAT
CTCAAGGTCCTGTTTTCCAGCAATGGGGGCGTCGTCAAAGGATTCAAGTTCTTCCAG
AAGGACCGCAAGATGGCACTGATCCAGATGGGCTCCGTGGAGGAGGCGGTCCAGGC
CCTCATTGACCTGCACAACCACGACCTCGGGGAGAACCACCACCTGCGGGTCTCCTT
CTCCAAGTCCACCATCTAGGGGCACAGGCCCCCACGGCCGGGCCCCCTGGCGACAA
CTTCCATCATTCCAGAGAAAAGCCACTTTAAAAACAGCTGAAGTGACCTTAGCAGAC
CAGAGATTTTATTTTTTTAAAGAGAAATCAGTTTACCTGTTTTTAAAAAAATTAAATCT
AGTTCACCTTGCTCACCCTGCGGTGACAGGGACAGCTCAGGCTCTTGGTGACTGTGG
CAGCGGGAGTTCCCGGCCCTCCACACCCGGGGCCAGACCCTCGGGGCCATGCCTTGG
TGGGGCCTGTGTCGGGCGTGGGGCCTGCAGGTGGGCGCCCCGACCACGACTTGGCT
TCCTTGTGCCTTAAAAAACCTGCCTTCCTGCAGCCACACACCCACCCGGGGTGTCCT
GGGGACCCAAGGGGTGGGGGGGTCACACCAGAGAGAGGCAGGGGGCCTGGCCGGC
TCCTGCAGGATCATGCAGCTGGGGCGCGGCGGCCGCGGCTGCGACACCCCAACCCC
AGCCCTCTAATCAAGTCACGTGATTCTCCCTTCACCCCGCCCCCAGGGCCTTCCCTTC
TGCCCCCAGGCGGGCTCCCCGCTGCTCCAGCTGCGGAGCTGGTCGACATAATCTCTGT
ATTATATACTTTGCAGTTGCAGACGTCTGTGCCTAGCAATATTTCCAGTTGACCAAATA
TTCTAATCTTTTTTCATTTATATGCAAAAGAAATAGTTTTAAGTAACTTTTTATAGCAAG
ATGATACAATGGTATGAGTGTAATCTAAACTTCCTTGTGGTATTACCTTGTATGCTGTTA
CTTTTATTTTATTCCTTGTAATTAAGTCACAGGCAGGACCCAGTTTCCAGAGAGCAGG
CGGGGCCGCCCAGTGGGTCAGGCACAGGGAGCCCCGGTCCTATCTTAGAGCCCCTGA
GCTTCAGGGAAGGGGCGGGCGTGTCGCCGCCTCTGGCATCGCCTCCGGTTGCCTTAC
ACCACGCCTTCACCTGCAGTCGCCTAGAAAACTTGCTCTCAAACTTCAGGGTTTTTTC
TTCCTTCAAATTTTGGACCAAAGTCTCATTTCTGTGTTTTGCCTGCCTCTGATGCTGGG
ACCCGGAAGGCGGGCGCTCCTCCTGTCTTCTCTGTGCTCTTTCTACCGCCCCCGCGTC
CTGTCCCGGGGGCTCTCCTAGGATCCCCTTTCCGTAAAAGCGTGTAACAAGGGTGTA
AATATTTATAATTTTTTATACCTGTTGTGAGACCCGAGGGGCGGCGGCGCGGTTTTTTA
TGGTGACACAAATGTATATTTTGCTAACAGCAATTCCAGGCTCAGTATTGTGACCGCG
GAGCCACAGGGGACCCCACGCACATTCCGTTGCCTTACCCGATGGCTTGTGACGCGG
AGAGAACCGATTAAAACCGTTTGAGAAACTCCTCCCTTGTCTAGCCCTGTGTTCGCTG
TGGACGCTGTAGAGGCAGGTTGGCCAGTCTGTACCTGGACTTCGAATAAATCTTCTGT
ATCCTC

125 antisense oligonucleotide (ASO) sequences were designed. The sequences of the ASO molecular are shown in Table 1,

Furthermore, the 125 ASO were chemically modified, specifically: monosulfation of all phosphodiester bonds in the nucleotides, and MOE modification of the 3′ and 5′ ends (i.e., methoxyethyl modification at the 2′ position of the bases) of the first 5 bases at each end. Among these, ASO2 (GTGGAAATATTGCTAGGCAC targeting PTBP1) and ASO3 (CCTATAGGACTATCCAGGAA, non-targeted to PTBP1) serve as positive and negative controls, respectively [1], with the same modification method as above.

TABLE 1
The information of ASO molecules (the underlined parts of the sequences in the
table are the loop regions of ASO)

| [symbol] | | | | |
|---|---|---|---|---|
| SEQ ID | sequence | | | |
| NO. | ID | ASO | startPos(+) | region |
| 1 | 20_30 | TTGGAGAACTGGATGTAGAT | 422 | EXON5 |
| 2 | 20_69 | TGGTTGGAGAACTGGATGTA | 425 | EXON5 |
| 3 | 20_74 | TTGTGGTTGGAGAACTGGAT | 428 | EXON5 |
| 4 | 20_78 | TGTGGTTGGAGAACTGGATG | 427 | EXON5 |
| 5 | 20_86 | GTGGTTGGAGAACTGGATGT | 426 | EXON5 |
| 6 | 20_90 | AACTGGATGTAGATGGGCTG | 416 | EXON5 |
| 7 | 20_83 | GGTTGGAGAACTGGATGTAG | 424 | EXON5 |
| 8 | 20_90 | AACTGGATGTAGATGGGCTG | 416 | EXON5 |
| 9 | 20_83 | GGTTGGAGAACTGGATGTAG | 424 | EXON5 |
| 10 | 19_150 | GTGGTTGGAGAACTGGATG | 427 | EXON5 |
| 11 | 20_71 | GAACTGGTTGTTCTTGGTGA | 693 | EXON7 |
| 12 | 20_110 | CTGGAACTGGTTGTTCTTGG | 696 | EXON7 |
| 13 | 20_63 | GGAACTGGTTGTTCTTGGTG | 694 | EXON7 |
| 14 | 20_72 | AACTGGTTGTTCTTGGTGAA | 692 | EXON7 |
| 15 | 20_89 | GCCTGGAACTGGTTGTTCTT | 698 | EXON7 |
| 16 | 20_18 | CAGCAGGCGTTGTAGATGTT | 779 | EXON8 |
| 17 | 20_37 | CGATGCGCAGCGTGCAGCAG | 793 | EXON8 |
| 18 | 20_53 | AGCGTGCAGCAGGCGTTGTA | 785 | EXON8 |
| 19 | 20_55 | AGCAGGCGTTGTAGATGTTC | 778 | EXON8 |
| 20 | 20_56 | CGCAGCGTGCAGCAGGCGTT | 788 | EXON8 |
| 21 | 20_75 | GCAGCAGGCGTTGTAGATGT | 780 | EXON8 |
| 22 | 20_77 | CAGGCGTTGTAGATGTTCTG | 776 | EXON8 |
| 23 | 20_111 | GCGCAGCGTGCAGCAGGCGT | 789 | EXON8 |
| 24 | 20_9 | TCGATGCGCAGCGTGCAGCA | 794 | EXON8 |
| 25 | 20_16 | AGTCGATGCGCAGCGTGCAG | 796 | EXON8 |
| 26 | 20_41 | GCGTGCAGCAGGCGTTGTAG | 784 | EXON8 |
| 27 | 20_60 | CAGCGTGCAGCAGGCGTTGT | 786 | EXON8 |
| 28 | 20_82 | GCAGGCGTTGTAGATGTTCT | 777 | EXON8 |
| 29 | 20_61 | TGCAGCAGGCGTTGTAGATG | 781 | EXON8 |
| 30 | 20_82 | GCAGGCGTTGTAGATGTTCT | 777 | EXON8 |
| 31 | 20_5 | GTCGATGCGCAGCGTGCAGC | 795 | EXON8 |
| 32 | 19 59 | CAGCAGGCGTTGTAGATGT | 780 | EXON8 |
| 33 | 20_21 | CCTGCATACGGAGAGGCTGA | 956 | EXON9 |
| 34 | 20_22 | TCCTGCATACGGAGAGGCTG | 957 | EXON9 |
| 35 | 20_39 | GCTCCTGCATACGGAGAGGC | 959 | EXON9 |
| 36 | 18_16 | CTTTGGGGTGTGACTCTC | 1165 | EXON10(1)/EXON |
| | | | | 11(17) |
| 37 | 19_16 | GCTTTGGGGTGTGACTCTC | 1165 | EXON10(1)/EXON |
| | | | | 11(18) |
| 38 | 20_46 | GGCTTTGGGGTGTGACTCTC | 1165 | EXON10(1)/EXON |
| | | | | 11(19) |
| 39 | 20_3 | GCTTTGGGGTGTGACTCTCT | 1164 | EXON10(2)/EXON |
| | | | | 11(18) |
| 40 | 20-3-1 | GCTTTGGGGTGTGTTTTTTTCTCTCT | 1164 | EXON10(2)/EXON |
| | | | | 11(18)/LOOP |
| 41 | 20-3-2 | GCTTTGGGGTGTGGGGTTCCCCTCTCT | 1164 | EXON10(2)/EXON |
| | | | | 11(19)/LOOP |
| 42 | 20-3-3 | GCTTTGGGGTGTGGCCTTTGGCCTCTCT | 1164 | EXON10(2)/EXON |
| | | | | 11(20)/LOOP |
| 43 | 20-3-4 | GCTTTGGGGTGTCGCCTTTGGCGTCTCT | 1164 | EXON10(2)/EXON |
| | | | | 11(21)/LOOP |
| 44 | 20-3-5 | GCTTTGGGGTGTGCGCCTTTGGCGTCTCT | 1164 | EXON10(2)/EXON |
| | | | | 11(22)/LOOP |
| 45 | 20-3-6 | GCTTTGGGGTGTGTTTTTTTTTTTTCTCT | 1164 | EXON10(2)/EXON |
| | | | | 11(23)/LOOP |
| 46 | 20-3-7 | GCTTTGGGGTGTGGGGTGGGTTGGGTGG | 1164 | EXON10(2)/EXON |
| | | GTCTCT | | 11(24)/LOOP |
| 47 | 20-3-8 | GCTTTGGGGTGTGGGGTGGGTTGGGTGG | 1164 | EXON10(2)/EXON |
| | | GCTCTCT | | 11(25)/LOOP |
| 48 | 20-3-9 | GCTTTGGGGTGTGGGGTTGGGTTTGGGTT | 1164 | EXON10(2)/EXON |
| | | GGGTTCTCTCT | | 11(26)/LOOP |
| 49 | 20-3-10 | GCTTTGGGGTGTGTTGGGTTGGGTTTGGG | 1164 | EXON10(2)/EXON |
| | | TTGGGTTCTCTCT | | 11(27)/LOOP |
| 50 | 20-3-11 | GCTTTGGGGTGTGTGCGTTTCGCTCGCTT | 1164 | EXON10(2)/EXON |
| | | TGCGTCTCTCT | | 11(28)/LOOP |
| 51 | 20_13 | AATAAAGAGGCTTTGGGGTG | 1173 | EXON11 |
| 52 | 20_19 | ATAAAGAGGCTTTGGGGTGT | 1172 | EXON11 |
| 53 | 20_20 | GAATAAAGAGGCTTTGGGGT | 1174 | EXON11 |
| 54 | 20_32 | GAGGCTTTGGGGTGTGACTC | 1167 | EXON11 |
| 55 | 20_50 | AGGCTTTGGGGTGTGACTCT | 1166 | EXON11 |
| 56 | 20_51 | AGAGGCTTTGGGGTGTGACT | 1168 | EXON11 |
| 57 | 20_54 | AAGAGGCTTTGGGGTGTGAC | 1169 | EXON11 |
| 58 | 20_58 | AGAATAAAGAGGCTTTGGGG | 1175 | EXON11 |
| 59 | V2_16_ | GGCTTTGGGGTGTGAC | 1169 | EXON11 |
| | 67 | | | |
| 60 | 19 49 | GAGGCTTTGGGGTGTGACT | 1168 | EXON11 |
| 61 | 19_51 | AATAAAGAGGCTTTGGGGT | 1174 | EXON11 |
| 62 | 20_38 | TTATTGAACAGGATCTTCAC | 1223 | EXON12 |
| 63 | 20_59 | CGTTCTCCTTCTTATTGAAC | 1234 | EXON12 |
| 64 | 20_81 | GCGTTCTCCTTCTTATTGAA | 1235 | EXON12 |
| 65 | 20_91 | CTTATTGAACAGGATCTTCA | 1224 | EXON12 |
| 66 | 20_107 | CTCCTTCTTATTGAACAGGA | 1230 | EXON12 |
| 67 | 20_64 | CCTTCTTATTGAACAGGATC | 1228 | EXON12 |
| 68 | 20_65 | GTTCTCCTTCTTATTGAACA | 1233 | EXON12 |
| 69 | 20_76 | TCTCCTTCTTATTGAACAGG | 1231 | EXON12 |
| 70 | V2_16_ | GTTCTCCTTCTTATTG | 1237 | EXON12 |
| | 80 | | | |
| 71 | 20_35 | CCGTTCAGGTGGCTCATGGC | 1292 | EXON12(1)/EXON |
| | | | | 13(19) |
| 72 | 20_88 | CGTTCAGGTGGCTCATGGCC | 1291 | EXON12(2)/EXON |
| | | | | 13(18) |
| 73 | 20-81-1 | GCGTTTTTTTTTTCCTTCTTATTGAA | 1235 | EXON12/LOOP |
| 74 | 20-81-2 | GCGTTCGCCTTTGGCGTCCTTCTTATTGAA | 1235 | EXON12/LOOP |
| 75 | 20-81-3 | GCGTTCTCCTTCTTTTTTTTTTTGAA | 1235 | EXON12/LOOP |
| 76 | 20-81-4 | GCGTTCTCCTTCTTCGCCTTTGGCGTTGAA | 1235 | EXON12/LOOP |
| 77 | 20-81-5 | GCGTTCTCCTTCTCGCCTTTGGCGTTGAA | 1235 | EXON12/LOOP |
| 78 | 20-81-6 | GCGTTCTCCCGCCTTTGGCGCTTATTGAA | 1235 | EXON12/LOOP |
| 79 | 20-81-7 | GCGTTCTCCGGCCTTTGGCCCTTATTGAA | 1235 | EXON12/LOOP |
| 80 | 20-81-8 | GCGTTCTCCTTTTTTTCTTATTGAA | 1235 | EXON12/LOOP |
| 81 | 20_1 | CGTGCAGCTTGTGCCCGTTC | 1306 | EXON13 |
| 82 | 20_2 | GTGCAGCTTGTGCCCGTTCA | 1305 | EXON13 |
| 83 | 20_4 | CCCGTGCAGCTTGTGCCCGT | 1308 | EXON13 |
| 84 | 20_7 | CCGTGCAGCTTGTGCCCGTT | 1307 | EXON13 |
| 85 | 20_8 | TCCCGTGCAGCTTGTGCCCG | 1309 | EXON13 |
| 86 | 20_36 | GTGCCCGTTCAGGTGGCTCA | 1296 | EXON13 |
| 87 | 20 48 | TGCAGCTTGTGCCCGTTCAG | 1304 | EXON13 |
| 88 | 20_49 | TTGTGCCCGTTCAGGTGGCT | 1298 | EXON13 |
| 89 | 20_52 | CTTGTGCCCGTTCAGGTGGC | 1299 | EXON13 |
| 90 | 20_57 | TGCCCGTTCAGGTGGCTCAT | 1295 | EXON13 |
| 91 | 20_23 | TTCCCGTGCAGCTTGTGCCC | 1310 | EXON13 |
| 92 | 20_26 | CCCGTTCAGGTGGCTCATGG | 1293 | EXON13 |
| 93 | 20_40 | CTTCCCGTGCAGCTTGTGCC | 1311 | EXON13 |
| 94 | 19_25 | TTCCCGTGCAGCTTGTGCC | 1311 | EXON13 |
| 95 | 20_80 | GAGGGCGGGATGTTGGAGAG | 1496 | EXON13(14)/EXO |
| | | | | N14(6) |
| 96 | 20_70 | AGGGCGGGATGTTGGAGAGG | 1495 | EXON13(15)/EXO |
| | | | | N14(5) |
| 97 | 20_34 | GGGCGGGATGTTGGAGAGGT | 1494 | EXON13(16)/EXO |
| | | | | N14(4) |
| 98 | 20_24 | GGCGGGATGTTGGAGAGGTG | 1493 | EXON13(17)/EXO |
| | | | | N14(3) |
| 99 | 20_15 | GCGGGATGTTGGAGAGGTGC | 1492 | EXON13(18)/EXO |
| | | | | N14(2) |
| 100 | 20_44 | CGGGATGTTGGAGAGGTGCA | 1491 | EXON13(19)/EXO |
| | | | | N14(1) |
| 101 | 20_45 | TGCCATCTTGCGGTCCTTCT | 1587 | EXON14(1)/EXON |
| | | | | 15(19) |
| 102 | 20_17 | GCGGTCCTTCTGGAAGAACT | 1578 | EXON14(10)/EXO |
| | | | | N15(10) |
| 103 | 19 63 | CGGTCCTTCTGGAAGAACT | 1578 | EXON14(10)/EXO |
| | | | | N15(9) |
| 104 | 20_98 | CGGTCCTTCTGGAAGAACTT | 1577 | EXON14(11)/EXO |
| | | | | N15(9) |
| 105 | 20_99 | CCATCTTGCGGTCCTTCTGG | 1585 | EXON14(3)/EXON |
| | | | | 15(17) |
| 106 | 20_100 | CATCTTGCGGTCCTTCTGGA | 1584 | EXON14(4)/EXON |
| | | | | 15(16) |
| 107 | 20_79 | ATCTTGCGGTCCTTCTGGAA | 1583 | EXON14(5)/EXON |
| | | | | 15(15) |
| 108 | 20_85 | CTTGCGGTCCTTCTGGAAGA | 1581 | EXON14(7)/EXON |
| | | | | 15(13) |
| 109 | 20_29 | TTGCGGTCCTTCTGGAAGAA | 1580 | EXON14(8)/EXON |
| | | | | 15(12) |
| 110 | 20_14 | TGCGGTCCTTCTGGAAGAAC | 1579 | EXON14(9)/EXON |
| | | | | 15(11) |
| 111 | 20_6 | CAGTGCCATCTTGCGGTCCT | 1590 | EXON15 |
| 112 | 20_11 | GTGCCATCTTGCGGTCCTTC | 1588 | EXON15 |
| 113 | 20_12 | GATCAGTGCCATCTTGCGGT | 1593 | EXON15 |
| 114 | 20_31 | AGTGCCATCTTGCGGTCCTT | 1589 | EXON15 |
| 115 | 20_33 | ATCAGTGCCATCTTGCGGTC | 1592 | EXON15 |
| 116 | 20_47 | TGGATCAGTGCCATCTTGCG | 1595 | EXON15 |
| 117 | 20_68 | TCAGTGCCATCTTGCGGTCC | 1591 | EXON15 |
| 118 | 20_101 | GGATCAGTGCCATCTTGCGG | 1594 | EXON15 |
| 119 | 20_25 | AGCCCATCTGGATCAGTGCC | 1603 | EXON15 |
| 120 | 20_42 | GAGCCCATCTGGATCAGTGC | 1604 | EXON15 |
| 121 | 20_87 | GCCCATCTGGATCAGTGCCA | 1602 | EXON15 |
| 122 | 20_25 | AGCCCATCTGGATCAGTGCC | 1603 | EXON15 |
| 123 | 20_62 | CCCATCTGGATCAGTGCCAT | 1601 | EXON15 |
| 124 | AS02 | GTGGAAATATTGCTAGGCAC | 2356 | EXON15 3UTR |
| 125 | ASO-2-1 | GTGGAAAAAAATATTGCTAGGCAC | 2356 | EXON15 |
| | | | | 3UTR/LOOP |
| 126 | ASO3 | CCTATAGGACTATCCAGGAA | — | — |

Note:
The information shown in Table 1 is determined based on the corresponding positions in the Ensembl database sequence.

### Example 2, Activities of Antisense Oligonucleotides (ASOs) Tested In Vitro Using Cell Models (Hela Human Cervical Cancer Cells)

In this example, the inhibitory effect of the ASO molecules shown in Table 1 on the expression of the PTBP1 gene (polyadenylate polypeptide binding protein 1) was verified. The specific experimental process is as follows:

(1) Preparation of suspension transfection reagent: ASO powder was dissolved in sterile water to a concentration of 10 μM. UltraFectin® transfection-specific serum-reduced culture medium (basalmedia, L530KJ) was used to dilute the 10 UM ASO stock solution to the desired concentration. The serum-reduced culture medium was used to dilute Lipofectamine 2000 transfection reagent (Invitrogen, 11668-019). Mix the diluted transfection reagent solution and ASO solution to prepare the pre-set concentration, prepare ASO transfection complexes at corresponding concentration gradients, mix by pipetting 3-5 times, and incubate at room temperature for 20 minutes.

(2) Cell processing: Observe the confluence rate of the Hela cell line under a microscope (>70%). Plate the cells one day before transfection at a density of 4×10³ cells/per well in a 96-well plate. Add 100 μl of DMEM medium containing 10% FBS to each well. Replace 50 μl of DMEM medium containing 10% FBS prior to transfection. Add the transfection complex prepared in step (1) with the pre-set ASO concentration to the 96-well plate and incubate at 37° C. in a 5% CO₂ incubator.

(3) After 24 hours, extract total RNA from the cells and detect PTBP1 mRNA expression in the cells via real-time quantitative PCR (Q RT-PCR). The PCR primers used for amplifying the housekeeping gene ACTB and PTBP1 are shown in Table 2:

TABLE 2
Primer and probe sequences

| Gene name | Sequence | Nucleotide sequence (5′-3′) |
|---|---|---|
| hPTBP1 | Forward primer | AGTCACACCCCAAAGCCTCT |
| | Reverse primer | CCATCTGCACTAGGGCGTTC |
| | Probe | ACGGTGACGTGCAGCGCGTGAAGAT |
| hActin | Forward primer | GACTACCTCATGAAGATCCTCACC |
| | Reverse primer | TCTCCTTAATGTCACGCACGATT |
| | Probe | CGGCTACAGCTTCACCACCACGGC |

Relative gene expression was calculated using the 2\hat{ }-ΔΔCT method (Livak method). The inhibition rate of antisense oligonucleotide mRNA expression levels was calculated using the following equation:

$$Inhibition rate = ( 1 - 2 \hat{} - Δ Δ CT ) \times 100 \% .$$

The experimental groups are as follows:
Cells treated with ASOs of various sequences as indicated by their respective numbers;
The blank control group (Blank) consists of cells not treated with any ASOs.

The efficiency of the ASO sequences listed in Table 1 in inhibiting PTBP1 mRNA in human cervical cancer HeLa cells is shown in Table 3-1&3-2.

The results indicate that the ASO sequences listed in Table 1 have a significant inhibitory effect on PTBP1 mRNA transcription.

TABLE 3-1
Inhibition rate of mRNA in Hela cells treated with the antisense oligonucleotides

| Sequence ID | | startPos(+) | 0.13 nM | 0.41 nM | 1.23 nM | 3.7 nM | 11.1 nM | 33.3 nM | 100 nM |
|---|---|---|---|---|---|---|---|---|---|
| Blank | | — | 0 | 0 | 0 | 0 | 0 | 0 | 0 |
| ASO2 (124) | | 2356 | 12% | 44% | 54% | 83% | 91% | 93% | 83% |
| ASO3 (126) | | — | 3% | 4% | −7% | −1% | −16% | −4% | −7% |
| 20-32 | (54) | 1167 | 20% | 41% | 30% | 59% | 84% | 97% | 98% |
| 20-81 | (64) | 1235 | −13% | 12% | 38% | 37% | 86% | 87% | 91% |
| 19-150 | (10) | 427 | −6% | 8% | −12% | 27% | 27% | 46% | 56% |
| 18-16 | (36) | 1165 | 14% | 32% | 45% | 81% | 83% | 79% | 100% |
| 19-16 | (37) | 1165 | 6% | 33% | 67% | 87% | 97% | 93% | 100% |
| 19-25 | (94) | 1311 | −4% | 18% | 32% | 47% | 73% | 86% | 75% |
| 19-49 | (60) | 1168 | 28% | 49% | 51% | 74% | 91% | 93% | 93% |
| 20-25 | (119) | 1603 | 5% | 10% | 31% | 69% | 84% | 75% | 72% |
| 20-62 | (123) | 1601 | 3% | 29% | 64% | 79% | 88% | 86% | 79% |
| 20-90 | (6) | 416 | 8% | 0% | 43% | 72% | 70% | 82% | 100% |
| 20-5 | (31) | 795 | 19% | 35% | 64% | 95% | 93% | 78% | 100% |

TABLE 3-2
Inhibition rate of mRNA in Hela cells treated
with the antisense oligonucleotides

| | | | |
|---|---|---|---|
| Sequnces ID | startPos(+) | 3.7 nM | 10 nM |

| | | | | |
|---|---|---|---|---|
| 20-36 | (86) | 1296 | 41% | 64% |
| 20-37 | (17) | 793 | 73% | 88% |
| 20-38 | (62) | 1223 | 42% | 61% |
| 20-44 | (100) | 1491 | 68% | 75% |
| 20-45 | (101) | 1587 | 67% | 76% |
| 20-46 | (38) | 1165 | 93% | 98% |
| 20-47 | (116) | 1595 | 84% | 88% |
| 20-48 | (87) | 1304 | 77% | 92% |
| 20-49 | (88) | 1298 | 55% | 81% |
| 20-50 | (55) | 1166 | 90% | 98% |
| 20-51 | (56) | 1168 | 81% | 90% |
| 20-52 | (89) | 1299 | 77% | 89% |
| 20-53 | (18) | 785 | 60% | 63% |
| 20-54 | (57) | 1169 | 66% | 80% |
| 20-55 | (19) | 778 | 62% | 74% |
| 20-56 | (20) | 788 | 87% | 92% |
| 20-57 | (90) | 1295 | 31% | 48% |
| 20-58 | (58) | 1175 | 56% | 69% |
| 20-59 | (63) | 1234 | 49% | 79% |
| 20-68 | (117) | 1591 | 74% | 73% |
| 20-69 | (2) | 425 | 37% | 71% |
| 20-70 | (96) | 1495 | 15% | 39% |
| 20-71 | (11) | 693 | 52% | 75% |
| 20-74 | (3) | 428 | 45% | 59% |
| 20-75 | (21) | 780 | 63% | 69% |
| 20-77 | (22) | 776 | 59% | 84% |
| 20-78 | (4) | 427 | 30% | 60% |
| 20-79 | (107) | 1583 | 50% | 62% |
| 20-80 | (95) | 1496 | 18% | 26% |

| | | | |
|---|---|---|---|
| ASO-2-1 (125) | 2356 | 55% | 82% |

| | | | | |
|---|---|---|---|---|
| 20-3-1 | (40) | 1164 | 70% | 78% |
| 20-3-2 | (41) | 1165 | 63% | 82% |
| 20-3-3 | (42) | 1166 | 58% | 80% |
| 20-3-4 | (43) | 1167 | 25% | 61% |
| 20-3-5 | (44) | 1168 | 58% | 84% |
| 20-3-6 | (45) | 1169 | 52% | 72% |
| 20-3-7 | (46) | 1170 | 25% | 24% |
| 20-3-8 | (47) | 1171 | 18% | 43% |
| 20-3-9 | (48) | 1172 | 22% | 4% |
| 20-3-10 | (49) | 1173 | 21% | 33% |
| 20-3-11 | (50) | 1174 | 22% | 26% |
| 20-3-12 | (51) | 1166 | 44% | 69% |

### Example 3, Activities of Antisense Oligonucleotides (ASOs) Tested In Vitro Using Cell Models (Hela Human Cervical Cancer Cells)

In this example, the inhibitory effect of the ASO molecules shown in Table 1 on the PTBP1 protein levels in HeLa cells was verified. The transfection reagent was prepared as in Example 2, and the experimental steps were as follows:
(1) A 6-well plate was used, with a cell density of 1×10⁵ cells per well;
(2) The concentration of the ASO molecule used to treat the cells was 10 nM;
(3) After 72 hours of ASO treatment, total protein was extracted from the treated Hela cells using RIPA lysis buffer (beyotime, P0013B) supplemented with PMSF (phenylmethylsulfonyl fluoride, beyotime, ST505) and protease inhibitors;
(4) Proteins were separated by sodium dodecyl sulfate-polyacrylamide gel electrophoresis and transferred to a PVDF membrane. After blocking with 5% nonfat milk powder, the membrane was incubated overnight at 4° C. with PTBP1 antibody (CST, 72669S) and actin antibody (Abcam, ab49900). The next day, the membrane was incubated with the secondary antibody (CST, 7074S) at room temperature for 1 hour. Protein expression levels were detected using an enhanced chemiluminescence detection system (GE Healthcare).

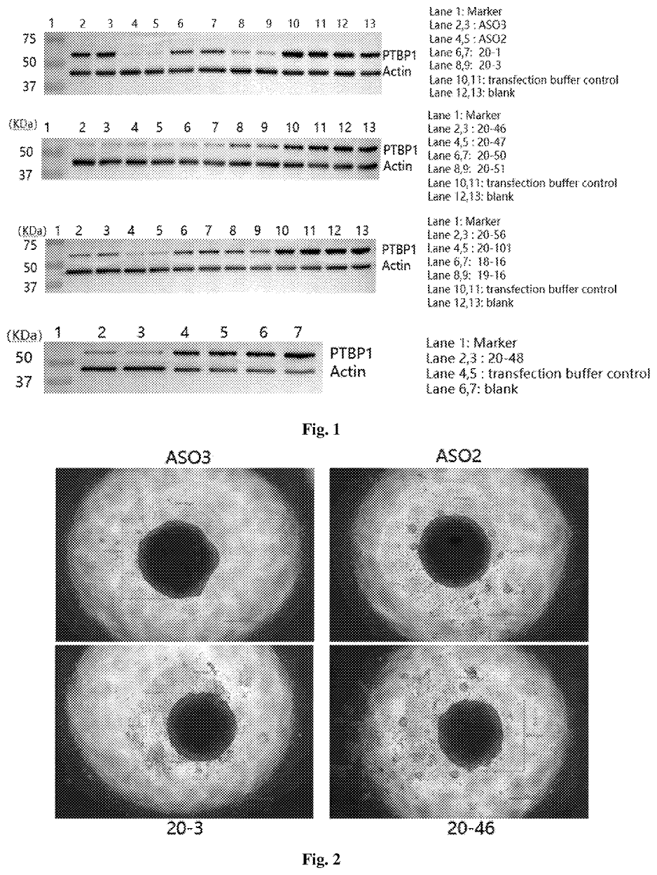

The inhibitory effects of some ASOs listed in Table 1 on PTBP1 protein expression in human cervical cancer HeLa cells are shown in FIG. 1 and Table 4. The results indicate that the ASO sequences listed in Table 1 have a significant inhibitory effect on PTBP1 protein expression.

TABLE 4
inhibition rate of PTBP1 protein

| | | |
|---|---|---|
| sequence ID | startPos(+) | [symbol] |

| | | | |
|---|---|---|---|
| 20-1 | (81) | 1306 | 48.43% |
| 20-3 | (39) | 1164 | 83.05% |
| 20-46 | (38) | 1165 | 89.52% |
| 20-47 | (116) | 1595 | 84.74 |
| 20-48 | (87) | 1304 | 85.85% |
| 20-51 | (56) | 1168 | 55.15% |
| 20-56 | (20) | 788 | 71.65% |
| 20-101 | (118) | 1594 | 80.73% |
| 18-16 | (36) | 1165 | 51.55% |
| 19-16 | (37) | 1165 | 50.47% |

### Example 4, Testing the Antiproliferation Effect of Antisense Oligonucleotides (ASOs) In Vitro (HCT116 Human Colon Cancer Cells)

In this example, the inhibitory effect of the ASO molecules shown in Table 1 on the proliferation of HCT116 cells has been verified.

The transfection reagent was prepared as in Example 2, and the experimental steps were as follows:
(1) A 24-well plate was used, with a cell density of 2×10⁴ cells per well;
(2) The concentration of the ASO molecules used to treat the cells was 30 nM;
(3) 24 hours after transfection, the cells were digested and seeded into a 96-well plate (Corning® 96-well black/transparent round-bottom spherical microplate, Corning, 4515) to form cell spheroids. After 120 hours, photographs were taken and the size was measured (since the spheroids were not very regular, the average of the length and width values was calculated to determine the radius, and the volume of the spheroids was calculated).

| | MS-ASO3 | MS-ASO2 | 20-3 (39) | 20-46 (38) |
|---|---|---|---|---|
| Length | 1041.8 | 927.05 | 884.78 | 821.36 |
| Width | 1002.54 | 917.99 | 869.68 | 905.91 |
| Radius | 511.08 | 461.26 | 438.61 | 431.82 |

The effects of some of the ASOs described in Table 1 on the proliferation of human colon cancer HCT116 cells are shown in FIG. 2, and Table 4 shows the inhibition rates calculated for the corresponding negative control ASO3. The results show that the ASO sequences shown in Table 1 have a significant inhibitory effect on the human colon cancer HCT116 cell line.

TABLE 4
Inhibition rates at different time points

| Time | ASO3 | ASO2 | 20-3 (39) | 20-46 (38) |
|---|---|---|---|---|
| 120 h | 0.00% | 26.49% | 36.8% | 39.69% |

### Example 5, Activities of Antisense Oligonucleotides (ASOs) Against Cells In Vitro Using Multicellular Models

In this example, The inhibitory effect of the ASO molecules provided in Example 1 on the expression of the polyadenylate binding protein 1 (PTBP1) gene in different cells was verified.

(1) Preparation of suspension transfection reagent: ASO powder was dissolved in sterile water to a concentration of 10 μM. UltraFectin® transfection-specific serum-reduced culture medium (basalmedia, L530KJ) was used to dilute the 10 UM ASO stock solution to the desired concentration. Serum-reduced culture medium was used to dilute Lipofectamine 2000 transfection reagent (Invitrogen, 11668-019). Mix the diluted transfection reagent and ASO solution to prepare the desired concentration, then prepare ASO transfection complexes at corresponding concentration gradients. Pipette 3-5 times to mix thoroughly, then incubate at room temperature for 20 minutes.

(2) Cell processing: Observe Hep3B, U138-MG, MDA-MB-231, HCT116, and A549 cell lines under a microscope to ensure a confluence rate >70%. Plate the cells the day before transfection at a density of 4×10³ cells/per well in a 96-well plate. Add 100 μl of DMEM medium containing 10% FBS to each well. Replace 50 μl of DMEM medium containing 10% FBS prior to transfection. Add the transfection complex prepared in step (1) with the set ASO concentration to the 96-well plate and incubate at 37° C. in a 5% CO₂ incubator.

(3) After 24 hours, extract total RNA from the cells and detect PTBP1 mRNA expression levels using real-time quantitative PCR (QRT-PCR). Calculate relative gene expression using the 2\hat{ }-ΔΔCT method (Livak method). The inhibition rate of antisense oligonucleotide mRNA expression levels is calculated using the following equation:

$$Inhibition rate = ( 1 - 2 \hat{} - Δ Δ CT ) \times 100 \% .$$

The experimental groups are as follows:
Cells treated with ASOs of various sequences as indicated by their respective numbers;
The blank control group (Blank) consists of cells not treated with any ASO.

The efficiency of PTBP1 mRNA inhibition by some of the ASO sequences described in Table 1 is shown in Tables 5-1 to 5-5.

TABLE 5-1
Inhibition rate of mRNA in Hep3B cells
treated with antisense oligonucleotides

| Sequnces ID | startPos(+) | 3.7 nM | 10 nM |
|---|---|---|---|
| Blank | — | −1 | −2 |
| ASO3(126) | — | −3 | 6 |
| ASO2(124) | 2356 | 69 | 82 |

| | | | | |
|---|---|---|---|---|
| 20-3 | (39) | 1164 | 45 | 82 |
| 20-46 | (38) | 1165 | 62 | 85 |
| 20-47 | (116) | 1595 | 22 | 75 |
| 20-50 | (55) | 1166 | 43 | 84 |
| 20-101 | (118) | 1594 | 36 | 78 |

TABLE 5-2
Inhibition rate of mRNA in U138-MG cells
treated with antisense oligonucleotides

| Sequnces ID | startPos(+) | 3.7 nM | 10 nM |
|---|---|---|---|
| Blank | — | 0 | −2 |
| ASO3(126) | — | 9 | −4 |
| ASO2(124) | 2356 | 49 | 66 |

| | | | | |
|---|---|---|---|---|
| 20-3 | (39) | 1164 | 46 | 64 |
| 20-46 | (38) | 1165 | 42 | 74 |
| 20-47 | (116) | 1595 | 23 | 58 |
| 20-50 | (55) | 1166 | 28 | 81 |
| 20-101 | (118) | 1594 | 25 | 61 |

TABLE 5-3
Inhibition rate of mRNA in MDA-MB-231 cells
treated with antisense oligonucleotides

| Sequnces ID | startPos(+) | 3.7 nM | 10 nM |
|---|---|---|---|
| Blank | — | −1 | −4 |
| ASO3(126) | — | −4 | −34 |
| ASO2(124) | 2356 | 40 | 52 |

| | | | | |
|---|---|---|---|---|
| 20-3 | (39) | 1164 | 45 | 63 |
| 20-46 | (38) | 1165 | 49 | 74 |
| 20-47 | (116) | 1595 | 27 | 62 |
| 20-50 | (55) | 1166 | 8 | 72 |
| 20-101 | (118) | 1594 | 4 | 58 |

TABLE 5-4
Inhibition rate of mRNA in HCT116 cells
treated with antisense oligonucleotides

| Sequnces ID | startPos(+) | 3.7 nM | 10 nM |
|---|---|---|---|
| Blank | — | −1 | −2 |
| ASO3(126) | — | −9 | −5 |
| ASO2(124) | 2356 | 65 | 77 |

| | | | | |
|---|---|---|---|---|
| 20-3 | (39) | 1164 | 38 | 70 |
| 20-46 | (38) | 1165 | 53 | 78 |
| 20-47 | (116) | 1595 | 38 | 61 |
| 20-50 | (55) | 1166 | 23 | 63 |
| 20-101 | (118) | 1594 | 17 | 58 |

TABLE 5-5
Inhibition rate of mRNA in A549 cells
treated with antisense oligonucleotides

| Sequnces ID | startPos(+) | 3.7 nM | 10 nM |
|---|---|---|---|
| Blank | — | 0 | −1 |
| ASO3(126) | — | −14 | −23 |
| ASO2(124) | 2356 | 86 | 86 |

| | | | | |
|---|---|---|---|---|
| 20-3 | (39) | 1164 | 74 | 86 |
| 20-46 | (38) | 1165 | 86 | 90 |
| 20-47 | (116) | 1595 | 64 | 69 |
| 20-50 | (55) | 1166 | 77 | 88 |
| 20-101 | (118) | 1594 | 60 | 72 |

### Example 6 Testing the Antisense Oligonucleotides (ASOs) Activities after being Modified with G-Quadruplex Modules In Vitro Cell Model (Hela Human Cervical Cancer Cells)

### Objective

In this embodiment, the inhibitory effect of the ASO molecules shown in Table 1 on the PTBP1 protein levels in Hela cells after modifying two different G4 chain sequences was verified. The transfection reagent was prepared as in Example 2. The structure and modification steps of the G4 chain are described in the applicant's prior application CN2021108831376. Specifically, the G4 chain structure was modified at the 3′ end of the nucleic acid,
for example,
20-3a modified form: 20-3 sequence GCTTTGGGGTGTGACTCTCT-G4 chain;
20-3b modified form: 20-3 sequence GCTTTGGGGTGTGACTCTCT-G4 chain (and the 3′ end of the G-4 chain is further modified with 2-methoxyethyl on the last three G bases);

In this example, the experimental steps are as follows:
(1) Use a 6-well plate with 1×10⁵ cells per well;
(2) The concentration of ASO molecules used to treat the cells was 10 nM;
(3) 72 hours after ASO treatment of the cells, total protein was extracted from the treated HeLa cells using RIPA lysis buffer (beyotime, P0013B) supplemented with PMSF (phenylmethylsulfonyl fluoride, beyotime, ST505) and protease inhibitors;
(4) Proteins were separated by sodium dodecyl sulfate-polyacrylamide gel electrophoresis and transferred to a PVDF membrane. After blocking with 5% nonfat milk, the membrane was incubated overnight at 4° C. with PTBP1 antibody (CST, 72669S) and actin antibody (Abcam, ab49900). The next day, the membrane was incubated with the secondary antibody (CST, 7074S) at room temperature for 1 hour. Protein expression levels were detected using an enhanced chemiluminescence detection system (GE Healthcare).

The inhibitory effect of the ASOs described in Table 1 on PTBP1 protein expression in human cervical cancer HeLa cells after modification of the G4 chain sequence is shown in FIG. 1 and Table 6.

The results indicate that specific ASOs, after modification of the G4 chain sequence, still exhibit a significant inhibitory effect on PTBP1 protein expression.

TABLE 6
PTBP1 protein inhibition rate

| sequence ID | startPos(+) | Protein inhibition rate (10 nM) |
|---|---|---|
| ASO2(124) | 2356 | 90.0% |

| | | | |
|---|---|---|---|
| 20-3 | (39) | 1164 | 78.8% |
| 20-3a | (39) | 1164 | 70% |
| 20-3b | (39) | 1164 | 63.9% |
| 20-46 | (38) | 1165 | 72.9% |
| 20-46a | (38) | 1165 | 53.4% |
| 20-46b | (38) | 1165 | 52.6% |

Finally, it should be noted that the above embodiments are only used to help the person skilled in the art to understand the essence of the present invention, and are not used to limit the scope of protection of the present invention.

### REFERENCES

[1] Maimon R, Chillon-Marinas C, Snethlage C E, Singhal SM, McAlonis-Downes M, Ling K, Rigo F, Bennett C F, Da Cruz S, Hnasko T S, Muotri A R, Cleveland D W. Therapeutically viable generation of neurons with antisense oligonucleotide suppression of PTB. Nat Neurosci. 2021 August; 24 (8): 1089-1099. doi: 10.1038/s41593-021-00864-y. Epub 2021 Jun. 3. PMID: 34083786; PMCID: PMC8338913.

1. An antisense oligonucleotide (ASO) molecules, wherein the ASO molecules specifically targets polypyrimidine tract binding protein 1 (PTBP1) mRNA at the following positions:
(1) positions 416-446 of EXON 5;
(2) positions 692-718 of EXON 7;
(3) positions 776-815 of EXON 8;
(4) positions 956-979 of EXON 9;
(5) positions 1160-1194 of EXON 10/EXON 11;
(6) positions 1223-1255 of EXON 12;
(7) positions 1291-1324 of EXON 12/EXON 13;
(8) positions 1305-1330 of EXON 13;
(9) positions 1491-1516 of EXON 13/EXON 14;
(10) positions 1577-1605 of EXON 14/EXON 15;
(11) positions 1587-1624 of EXON 15;
(12) positions 2356-2376 of EXON 15 3′UTR;
wherein the PTBP1 mRNA sequence is as shown in SEQ ID NO: 127.

2. The ASO molecule according to claim 1, wherein the ASO molecule specifically targets the PTBP1 gene mRNA at the following positions: positions 1164-1185 of EXON 10/EXON 11.

3. The ASO molecule according to claim 1, wherein the molecular length of the ASO molecule is 15 to 20 nucleotides.

4. The ASO molecule according to claim 11, wherein the sequence of the ASO molecule is selected from: SEQ ID NO:6, 20, 31, 36-41, 55-56, 60, 87, 89, 100-101, 116-119, and 123.

5. The ASO molecule according to claim 11, wherein the ASO molecule comprises at least one modified nucleotide.

6. The ASO molecule according to claim 5, wherein the modified nucleotide is selected from at least one of the following: 5′-phosphorothioate nucleotides, 5-methylcytosine nucleotides, 2′-O-methyl modified nucleotides, 2′-O-2-methoxyethyl modified nucleotides, 2′-fluoro modified nucleotides, 3′-aza substituted nucleotides, 2′-deoxy-2′-fluoro modified nucleotides, 2′-deoxy modified nucleotides, locked nucleic acids, abasic nucleotides, 2′-amino modified nucleotides, morpholino nucleotides, peptide nucleic acids, and phosphoramidates.

7. The ASO molecule according to claim 11, wherein all nucleotides in the ASO molecule are 5′-thio-phosphate-linked nucleotides, and the 5′ and 3′ ends each have five nucleotides modified with 2′-O-methoxyethyl (MOE).

8. A pharmaceutical composition comprising a therapeutically effective amount of an ASO molecules according to claim 11 and a pharmaceutical acceptable excipient;
wherein the pharmaceutical composition comprises an aqueous gel formulations or an injectable formulation; and
wherein the pharmaceutical composition is formulated for an administration route comprising local, intravenous, intramuscular, subcutaneous, or intradermal.

9. Use of the ASO molecule according to claim 11 in inhibiting PTBP1 gene expression.

10. A method of treating a disease caused by abnormal expression of PTBP1 gene, the method comprising administering the ASO molecule according to claim 11 to a subject in need thereof; wherein the disease comprises a tumor, Parkinson's disease, Alzheimer's disease, or Huntington's disease.

11. The ASO molecule according to claim 1, wherein the sequence of the ASO molecule is selected from: SEQ ID NO:1 to 123.

12. The ASO molecule according to claim 4, wherein the sequence of the ASO molecule is selected from: SEQ ID NO: 38 and 39.
